(19) United States
(12) Patent Application Publication
(10) Pub. No.: US 2022/0336080 A1
(43) Pub. Date: Oct. 20, 2022

(54) NON-INVASIVE ROBOTIC THERAPY SYSTEM

(71) Applicant: David R. Monteverde, San Diego, CA (US)

(72) Inventor: David R. Monteverde, San Diego, CA (US)

(21) Appl. No.: 17/722,365

(22) Filed: Apr. 17, 2022

Related U.S. Application Data
(60) Provisional application No. 63/175,681, filed on Apr. 16, 2021.

(51) Int. Cl. G16H 20/40; G06T 19/20; G06T 7/246; G06V 20/64; G06T 17/00; G06T 7/55; G05B 19/4155; B25J 9/16; A61H 39/02

(52) U.S. Cl. CPC G16H 20/40; G06T 19/20; G06T 7/251; G06V 20/64; G06T 17/00; G06T 7/55; G05B 19/4155; B25J 9/1697; A61H 39/02; G06T 2210/56; G06T 2207/10024; G06T 2207/10028; G06T 2210/41; G06T 2219/2016; G06T 2219/2012; G05B 2219/50391; A61H 2201/1659; A61H 2201/5092

(57) ABSTRACT

Disclosed herein is a robotic system comprising a robotic arm, an end effector coupled to a distal end of a robotic arm, one or more cameras, and a processors configured to construct a three-dimensional (3D) model of a user based on received data from the one or more cameras, identify automatically a target therapy point on the user based on the constructed 3D model, and actuate the end effector to apply a therapy to the target therapy point.

### CROSS-REFERENCE TO RELATED APPLICATIONS

This application claims the benefit under 35 U.S.C. § 119(e) of US Provisional Application No. 63/175,681, filed Apr. 16, 2021, which is incorporated by reference in its entirety.

### TECHNICAL FIELD

This application relates generally to medical robots, and particularly to robotic systems that perform non-invasive therapies on the human body.

### BACKGROUND

Current medical treatments rely predominantly on prescription drugs and surgery. These have very high rates of effectiveness but are often coupled with serious risks, such as drug side-effects and surgical complications. In the face of these risks there is a compelling (though less well-known) alternative to treat various kinds of ailments: acupuncture. Acupuncture is a form of therapy in which needles are used to stimulate specific points on the body, to relieve targeted conditions. Despite its use of needles, acupuncture is fundamentally non-invasive. This is because treatment is predominantly restricted to the body surface, and the needles themselves are usually quite thin. Currently, acupuncture is mostly performed by trained chiropractors. It may be desirable to have robotic systems that can deliver accurate and non-invasive treatment, such as acupuncture.

### SUMMARY

One or more aspects of the disclosed technology relate to robotic systems that perform non-invasive therapies on human body. The system integrates methods from multiple fields to locate anatomy-based points on the human body, and then applies non-invasive therapies at these points using robotic mechanisms. Methods utilized by the system relate to fields, including biomechanics, computer vision, artificial intelligence, and robotics.

In some variations, a robotic system comprises a robotic arm, an end effector coupled to a distal end of a robotic arm, one or more cameras, and a processors configured to construct a three-dimensional (3D) model of a user based on received data from the one or more cameras, identify automatically one or more target therapy points on the user based on the constructed 3D model, and actuate the end effector to apply a therapy to the target therapy points.

In some variations, a computer-implemented method comprises receiving, by a processor, data about a user from one or more sensors; constructing a three-dimensional (3D) model of the user based on the received data from the one or more sensors; identifying, by the processor, a target point on the user based on the constructed 3D model; and actuating an end effector to apply therapy to the target point.

In some variations, an apparatus comprises one or more sensors and a control unit configured to construct a 3D model of a user based on received data from the one or more sensors; identify a therapy point on the user based on the constructed 3D model; and actuate an end effector to treat the target point.

### BRIEF DESCRIPTION OF THE DRAWINGS

Certain features of the subject technology are set forth in the appended claims. However, for purpose of explanation, several embodiments of the subject technology are set forth in the following figures.

FIG. 1 is a 3D rendering of an example robotic system in accordance with one or more aspects of the subject technology.

FIG. 2 is a block diagram illustrating example operations of the robotic system in accordance with one or more aspects of the subject technology.

FIG. 3 is a diagram illustrating an example process of user model scaling in accordance with one or more aspects of the subject technology.

FIG. 4 is a diagram illustrating an input and output of image synthesis and 2D key-point localization in accordance with one or more aspects of the subject technology.

FIG. 5 is a block diagram illustrating an example process of image synthesis and 2D key-point localization in accordance with one or more aspects of the subject technology.

FIG. 6 is a diagram illustrating example degrees of freedom (DOFs) in 3D motion tracking in accordance with one or more aspects of the subject technology.

FIG. 7 is a diagram illustrating an example optimization process of 3D motion tracking in accordance with one or more aspects of the subject technology.

FIG. 8 is a diagram illustrating an example process of matching skin mesh to sensed surface in 3D motion tracking in accordance with one or more aspects of the subject technology.

FIG. 9 is a diagram illustrating an example method of therapy point mapping in accordance with one or more aspects of the subject technology.

FIG. 10 is a flowchart illustrating example operations of the robotic system in accordance with one or more aspects of the subject technology.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced using one or more implementations. In one or more instances, structures and components are shown in block diagram form in order to avoid obscuring the concepts of the subject technology.

Sometimes acupuncture is thought of as whimsical Eastern medicine, lacking any scientific footing. However, this is an unfounded misconception. Both the National Institutes of Health (NIH) and the World Health Organization (WHO) have reported scientific consensus that there is positive, incontrovertible evidence for the effectiveness of acupuncture in a number of conditions. Acupuncture therapy is commonly user for 1) pain treatments (e.g., back pain, arthritis pain, and migraines) as an alternative to opioids, corticosteroids or triptans, 2) surgery avoidance (e.g., kidney stones), and 3) mental health (e.g., depression or PTSD).

Acupuncture therapy can be applied with or without any needle penetration at all. Stimulation of therapy points, or acupoints, can be done in the form of acupressure (applying pressure with fingertips or other blunt objects), electric acupuncture, and even laser acupuncture (i.e., photo-acupuncture). In general, non-invasive treatments, such as acupuncture, can be referred to as non-invasive contact therapies, and have been prescribed to people of all ages. But notably, because of their low risk, simplicity of treatment, and ease of delivery, they are particularly attractive given current global demographic and socio-economic trends such as an aging world population.

An increasingly older world population demands ever growing and more affordable healthcare services. According to projections from the United Nations, in China alone the population aged 60 and above will increase by about 125 million (roughly equivalent to the entire population of Japan) over the next decade, while the total population will remain about the same. This trend is also present, although a bit less dramatically, in most industrialized countries. This means that the ratio of healthcare practitioners to healthcare consumers will rapidly decrease, and thus produce an explosive global demand for more accessible healthcare and wellness services.

In non-traditional markets for acupuncture, such as Western countries, its use has been rising steadily over the last few decades. In the United States, acupuncture treatment is covered by several large health insurance plans including Aetna, Blue Cross, and MHS (U.S. military healthcare services). Additionally, acupuncture coverage is mandated by state law in several states, such as California, Texas, Florida, Nevada, Montana, Maine and Virginia. Conspicuously, being of Asian descent is not a significant predictor of acupuncture use in the U.S. market.

Disclosed herein is a robotic system that can perform non-invasive therapies on the human body. The system integrates multidisciplinary methods to identify anatomy-based therapy points on human body and applies non-invasive therapies using robotic mechanisms. Methods utilized by the system relate to fields including biomechanics, computer vision, artificial intelligence and robotics.

The system utilizes a sensor array together with a human biomechanical model to build a personalized model of the underlying anatomy for the user based on observations of the user's skin surface in a few different poses. It constructs this user-specific biomechanical model by transferring elements of an anatomical template to the specific size and kinematics of the user. The system then maps therapy points to the anatomy model of the user and applies prescribed therapies using robotic apparatus. The system can follow user movement while treating the therapy points. The system also includes end effector to apply desired therapies, such as pressure, vibration, heat, electricity, laser, acoustic waves, needling, moxibustion, and vacuum cupping, at the therapy points.

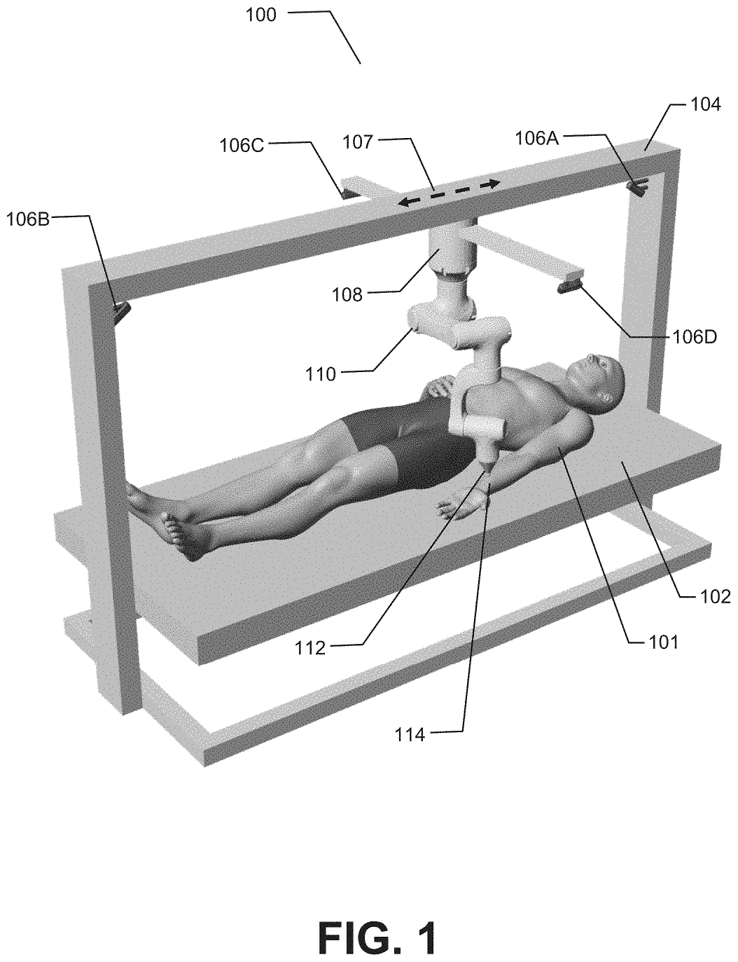

FIG. 1 is a 3D rendering of an example robotic system 100 in accordance with one or more aspects of the subject technology. The hardware components of the robotic system 100 includes a table 102, a frame 104 (with an axial rail 107 under the top frame), a sensor array 106 of four cameras 106A, 106B, 106C and 106D, a pedestal 108, a robotic arm 110, and an end-effector 112 coupled to the distal end of the robotic arm 110. Note that the robotic system may include more or less and/or different components other than the table, frame, sensor array, pedestal, arm, and the end-effector illustrated in FIG.1, and may be in different arrangement. For instance, sensor array 106 may comprise less than or more than four sensors of the same or different types, and be positioned in various parts of the robotic system 100.

As shown in FIG. 1, The user 101 rests on the therapy table 102 while receiving audible messages from the system, such as instructions to adopt a certain pose. The user 101's body is observed by the sensor array 106, which may comprise of four RGB-D (color and depth) cameras 106A, 106B, 106C and 106D. The cameras 106A and 106B are located near the user's head and feet, respectively, and are fixed to the frame 104. The cameras 106C and 106D are attached to robot pedestal 108, which moves along the top of the frame 104 via the axial rail 107. Attached to the lower part of pedestal 108 is the robot arm 110. The end-effector 112 is coupled to the distal end of robot arm 110, which can apply a therapy modality at a target therapy point 114 on the user. End-effector 112 may comprise apparatus to deliver one or more of the therapy modalities, such as pressure, vibration, heat, electricity, laser, acoustic waves, needling, moxibustion, and vacuum cupping. Components to support production and/or delivery of therapy modalities can also be housed in other parts of the system.

Axial motion of robot pedestal 108 along the top of frame 104 facilitates positioning of the system relative to therapy point TP. The design is to maximize unobstructed views of user 101 while minimizing occlusions caused by robot arm 110. Axial motion of the robot pedestal 108 (e.g., moving to either end of the frame 104) also facilitates physical access when the user enters or exits the system.

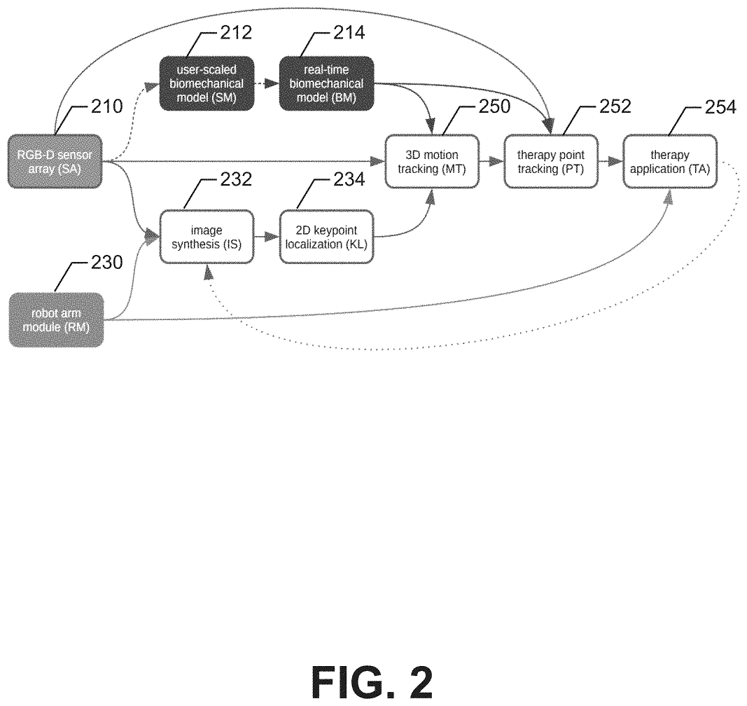

FIG. 2 is a block diagram illustrating example operations of the robotic system in accordance with one or more aspects of the subject technology. Example functioning components of the system 100 include sensing block 210, robotics block 230, scaling block 212, modeling block 214, imaging block 232, therapy point localization block 234, 3D motion tracking block 250, therapy point tracking 252, and therapy application block 254. The sensing block 210 is associated with the sensor array 106 (i.e., cameras 106A-D), while robotics block 230 is associated with pedestal 108, robotic arm 110, and end-effector 112 of the robotic system 100. The rest of the functional components are associated with a process or a computer (not shown in FIG.1), which controls the operations through software modules. Note that the robotic system 100 may include more or less and/or different functional components than those shown in FIG. 2.

As shown in FIG. 2, initial steps in system operation include the construction of a scaled biomechanical model of the user 101, performed by scaling block 212. The scaled model may be constructed before any therapy sessions and can be re-applied in subsequent sessions. The model construction comprises mapping a detailed biomechanical template of skeleton, musculature, skin and fat layers to the specific size and morphology of the user.

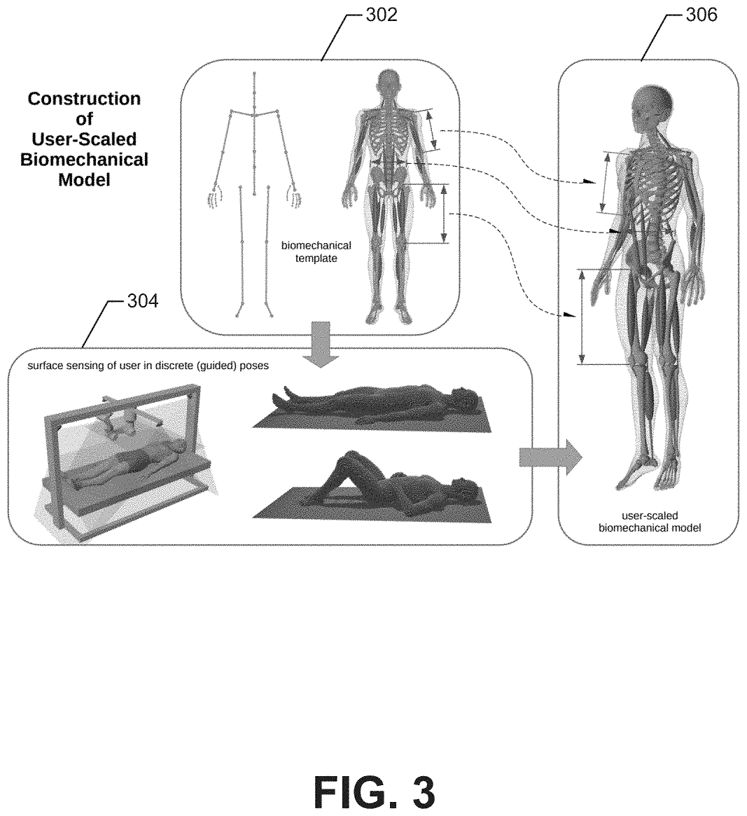

FIG. 3 is a block diagram illustrating an example process of user model scaling in accordance with one or more aspects of the subject technology. The process begins with templates of human body in block 302. Next the system captures a few (typically 2 to 5) different poses of the user with the depth cameras 106 (e.g., guided by the audio cues or voice commands) in block 304, and adjust bone sizes, musculature, and adiposity (i.e., subcutaneous fat) of the template to match the user's body surface (e.g., a point cloud) captured by the RGB-D cameras 106. The system can then construct a scaled biomechanical model of the user in block 306. The process described herein may involve concurrent multi-pose optimization and can be computationally intensive because it is built on physical and biomedical model of human body, including tasks, such as keeping track of volume collisions of different body parts. However, utilizing high-end processors currently available on the market, it may normally take several seconds to complete the process.

In some aspects, the process of scaling block 212 illustrated in FIG. 3 based on templates can be initialized manually via interactive selection of approximately 15 landmark or key-points around human body. However, the system can also perform autonomous key-point detection using artificial intelligence (AI). A brief description of AI-based key-point detection can be found in the following paragraphs.

Referring back to FIG. 2, once scaling template models for the user is complete at scaling block 212, modeling block 214 can construct a real-time biomechanical model of the user. The real-time model may retain the bone sizes, skin surface mesh and centerlines of muscles and tendons from scaling block 212. Furthermore, instead of using detailed volumetric information of muscles, modeling block 214 can manage the real-time model with muscle locations by constraining movement of tendon centerlines around wrapping surfaces that are fixed to bones to improve the model.

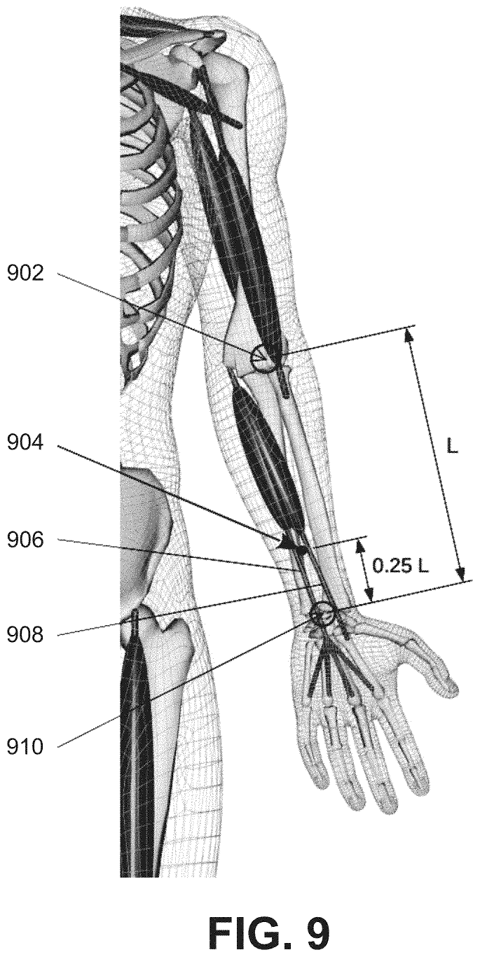

In contrast to the scaling block 212, which relies on physics-based methods for tasks such as skin deformation, the modeling block 214 may not need to. Instead, modeling block 214 can use geometric skinning techniques, such as linear blend skinning and dual quaternion skinning. These simplifications ensure modeling block 214 performs much faster than scaling block 212, thus more amenable for real-time tracking of the user on the table during therapy. In addition, the modeling block 214 can also be augmented through a mapping between therapy point locations and musculoskeletal geometry of human body. An example of the geometry mapping of therapy point is shown in FIG. 9 and described in the following paragraphs.

Once the real-time biomechanical model of the user is established by the modeling block 214, a therapy session can take place. During the therapy session, a continuous loop of sequenced steps can be performed by imaging block 232, therapy-point (or key-point) localization block 234, 3D motion tracking block 250, therapy point tracking 252, and therapy application block 254, as shown in FIG. 2.

The therapy session starts with the imaging block 232, which can produce a synthetic 2D image of the user 101. The synthetic image can then be fed to therapy point localization block 234 to identify the therapy points on the user in 2D image.

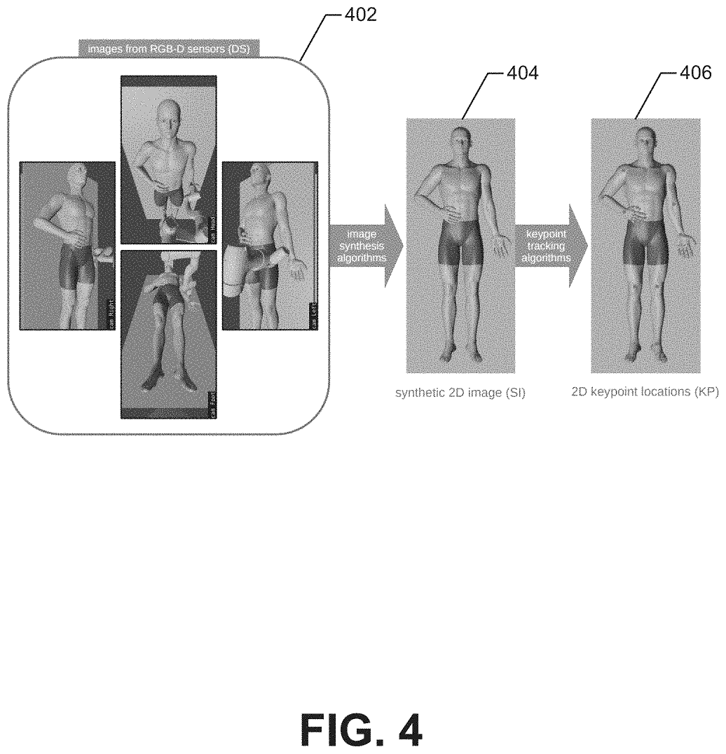

FIG. 4 illustrates the input and output of imaging block 232 and therapy point localization block 234 in accordance with one or more aspects of the subject technology. As shown in FIG. 4, input images 402 to imaging block 232 include 4 images captured by the imaging sensor array 106. A synthetic image 404 of the user 101 can be generated from input images 402. The therapy-point localization block 234 can localize therapy point on the image 406.

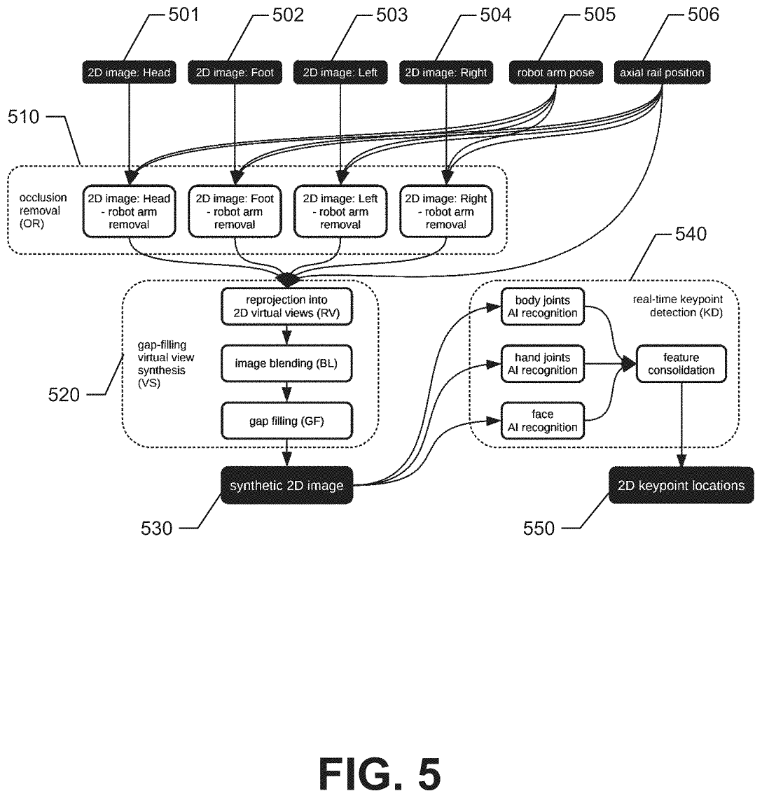

FIG. 5 is a block diagram illustrating a more detailed process of image synthesis and 2D key-point localization in accordance with one or more aspects of the subject technology. The imaging block 232 takes the input images 501, 502, 503 and 504 taken by the cameras 106A, 106B, 106C and 106D, respectively, as well as the robotic arm pose 505 and axial rail position of pedestal 108, and performs occlusion removal step 510. Occlusion removal step 510 comprises removing pixels of robotic arm 110 and end-effector 112 from each of the four input images 501-504. For example, pixel occupied by robot arm 110 and end-effector 112 can be inferred from input pose of the robot arm 110 (known by internal sensors/encoders of the robot arm and the axial rail) and 3D surface sensed by the depth cameras 106. Pixels corresponding to a spatial match (within a defined confidence level) can be removed from all four input images.

Image synthesis process further includes a virtual view synthesis with gap-filling 520. The method may involve reprojection of the user into a virtual view, image blending and gap filling, as shown in FIG. 5. In particular, virtual synthesis 520 may re-project the input views into virtual views from a common viewpoint. The virtual view may originate from a viewpoint that is different from the actual viewpoints of the depth cameras. Each 2D pixel in a respective input image corresponds to a line in 3D space and depth information constrains that line to a single point, establishing a correspondence between pixels and 3D points. Thus, a 3D point cloud encoded in each input image from cameras 106 enables the system to render scene geometry from an arbitrary viewpoint. The virtual viewpoint is normally selected to be farther away from the user than any of the depth cameras 106 to reduce perspective distortion. Also, the virtual viewpoint is typically in a region directly above the therapy table, but can vary, to maximize visibility of key-points close to the therapy point.

Next, image blending module combines the reprojected views into a synthesized virtual view. The blending process can include brightness adjustments, to reduce inconsistent brightness and discontinuous colors. Finally, necessary gap-filling is performed because the blended virtual view may include parts of the scene not visible in any of the input images, caused by the limited number of discrete viewpoints. Several standard techniques exist for gap-filling, including inpainting methods, which use texture from adjacent regions to fill gaps in the blended synthetic image.

Synthetic image 530 thus generated can then be input to key-point detection block 540, which may use real-time 2D key-point detection methods, such as machine learning algorithms trained on large databases of manually annotated images with information of interest (e.g., locations of key-points). For example, AI models with an artificial neural network (ANN) trained on an extensive number of images to detect key-points in real-time. The 2D key-point detection method can perform real-time inference of therapy points on body key-points (e.g., shoulder, elbows, hips, and knees), hand key-points (e.g., wrist, finger joints, and fingertips), foot key-points, and facial key-points (e.g., eyes, nose, and ears), and integrate the inference into a consistent body arrangement. The method is robust with respect to occluded (blocked from view) body parts and can also determine whether the user is positioned face-up or face-down. In short, the output of block 540 is an annotate 2D image 550 with key-point locations marked.

Referring back to FIG. 2, next step in system operation is 3D motion tracking by block 250, which takes input of skeleton kinematics and skin mesh from modeling block 214, captured image and depth info (i.e. surface point cloud) of the user from RGB-D sensor array 210, and 2D key-point locations detected by key-point localization block 234.

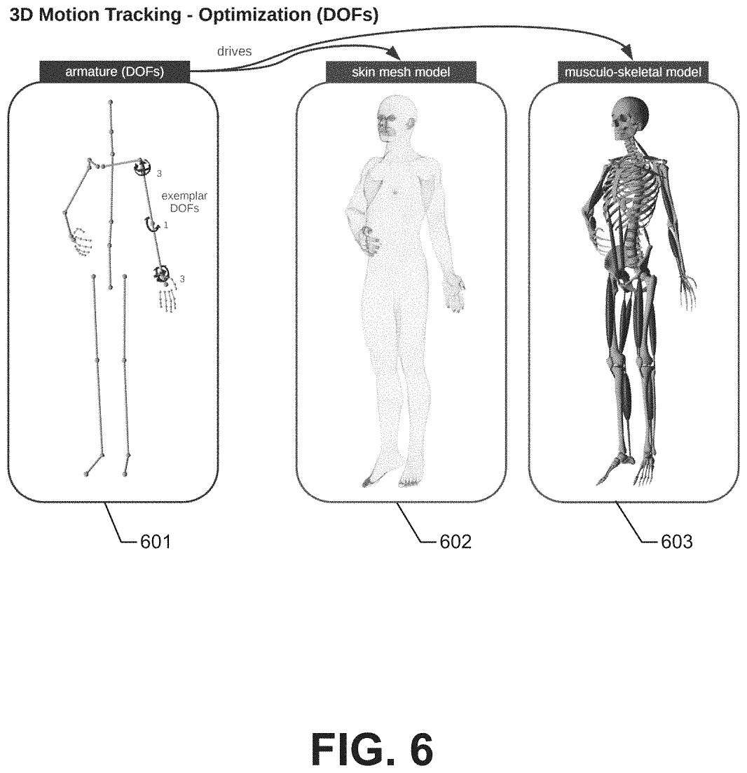

In some aspects, motion tracking performed by block 250 concerns optimization variables that correspond to degrees of freedom (DOFs) of the skeletal armature in real-time biomechanical model, as illustrated in FIG. 6. The skeletal armature 601 is essentially an abstraction for the kinematics of musculoskeletal model 603, which is a component of the real-time biomechanical model generated by modeling block 214 in FIG. 2, alongside other components, such as the skin mesh model 602.

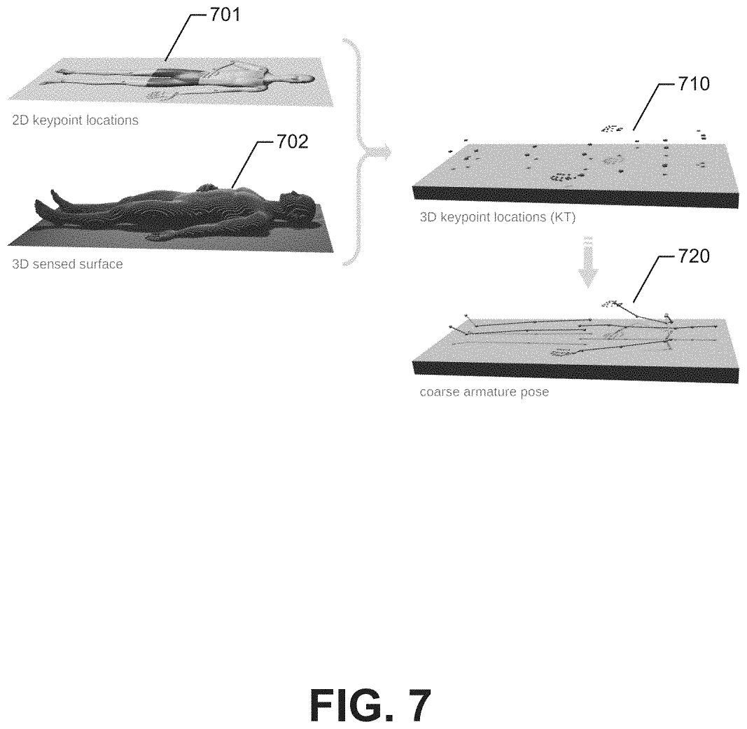

FIG. 7 is a diagram illustrating an example optimization process of 3D motion tracking in accordance with one or more aspects of the subject technology. For instance, the motion tracking can map each 2D key-point 701 (e.g., 2D image of key-point locations 550 in FIG. 5) onto the sensed point cloud surface 702 (e.g., surface point cloud from block 210 in FIG. 2), and then projects it along the surface normal by a fixed distance (pointing inwards) to yield the 3D key-point map 710. Each of the fixed distances projected inwards from the surface depends on the individual key-point and expresses a joint radius for that key-point (this data is included from the real-time biomechanical model from modeling block 214).

Note that since the key-point detection relies on machine learning trained from manually annotated 2D images of anatomical landmarks, joint locations marked by AI inference may not be highly accurate. However, once these coarse locations 720 are known, the tracking algorithm can further optimize the armatures's DOFs for the key-point localization.

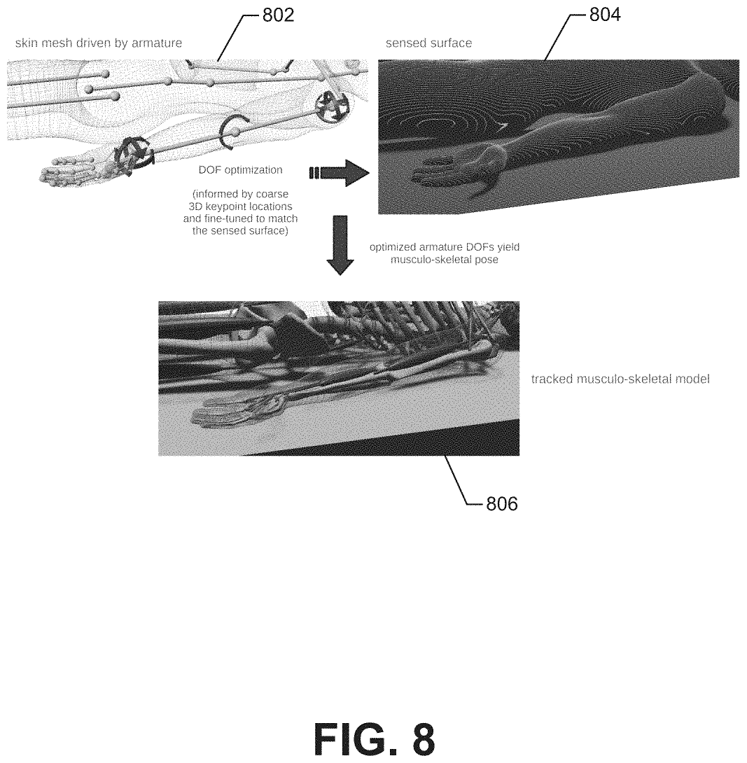

FIG. 8 is a diagram illustrating an example process of matching skin mesh to sensed surface in 3D motion tracking in accordance with one or more aspects of the subject technology. For example, armatures's DOFs 802 can be further fine-tuned to match the skin mesh of the biomechanical model against the point cloud surface sensed 804 by the depth cameras, thus yielding highly accurate tracking 806. Therefore, the 3D motion tracking comprises an optimization that concurrently minimizes distances between 1) the skeletal armature joints and 3D key-points, as well as 2) skin mesh and 3D surface point cloud.

Referring back to FIG. 2, after 3D motion tracking, block 252 continues to map therapy points with respect to bony reference points and/or muscle locations within the musculoskeletal model and projected onto the skin surface. The mapping can be based on anatomical landmarks and geometric relationships, similar to the standardized descriptions provided by the WHO for more than 350 acupoints.

FIG. 9 is a diagram illustrating an example method of therapy point mapping in accordance with one or more aspects of the subject technology. For instance, the mapping of acupoint PC-5 904 can be accomplished as follows: 1) based on a distance L between elbow 902 and wrist 910, 2) located ¼ L from the wrist 910, and 3) halfway between tendon of Palmaris Longus muscle 908 and tendon of Flexor Carpi Radialis muscle 908.

Referring back to FIG. 2, the last step in the operation is therapy application by block 254. The robotics block 230 controls the robot arm 110 and the axial rail 107 to position the tip of the end-effector 112 at a localized therapy point 114 (see FIG. 1). The main axis of the end-effector is typically normal to the skin surface at the therapy point, but this can vary according to the therapy. The end-effector applies one or more therapy modalities at the point (e.g., pressure, laser, or heat), and then continues to the next therapy point in a prescribed sequence, where it applies the next prescribed therapy modality.

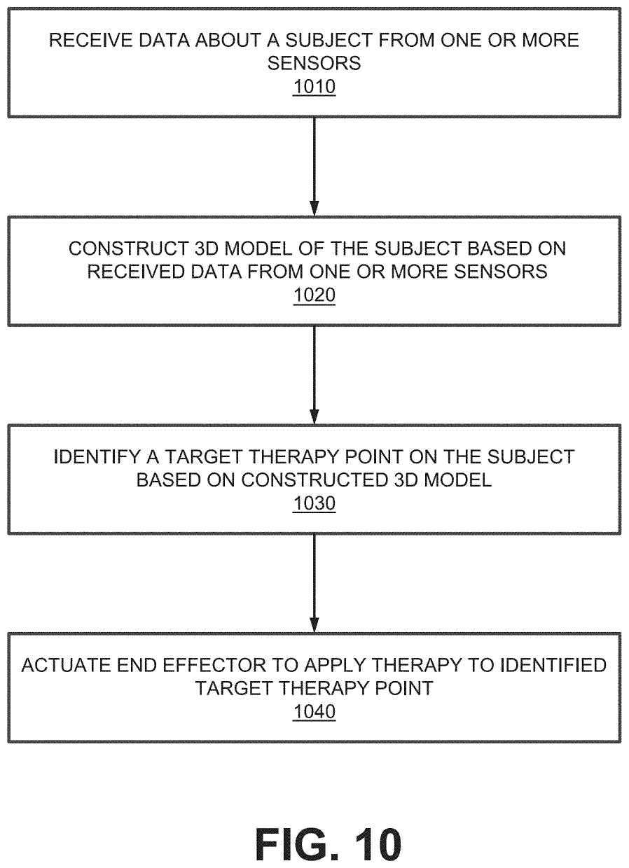

FIG. 10 is a flowchart illustrating example operations of the robotic system in accordance with one or more aspects of the subject technology. The system first receives 1010 data about a user from one or more sensors, such as color and depth images of user 101 captured by the sensor array 106 shown in FIG. 1.

The system also constructs 1020 a 3D model of the subject based on received data from the one or more sensors. For instance, the scaling block 212 and modeling block 214 in FIG. 2 can map a detailed biomechanical template of skeleton, musculature, skin and fat layer to the specific size and morphology of the user in real-time.

Next, the system identifies a target therapy point (or key-point) on the subject based on the constructed 3D model of the user. For example, imaging block 232 and key-point localization block 234 in FIG. 2 can produce a synthetic image of the user without any occlusion and identify therapy points on the user from the synthetic image. Next, 3D motion tracking block 250 and therapy point tracking 252 can map the therapy points onto the surface point cloud of the user. The system then actuates end effector to apply therapy to the identified target therapy point.

Users may obtain customized therapy sessions in various ways, such as: 1) provided by the system (via kiosk, terminal, or mobile device), in which user can select menu options or automated recommendation based on user needs, such as certain common conditions, and generic treatments (e.g., relaxation against stress); 2) prescribed by therapy expert, such as an acupuncturist either remotely via videotelephony or in-person. Prescription instructions may include: 1) a set of therapy points or acupoints, 2) a sequence and timing of acupoint treatment, and 3) treatment modality per acupoint, such as pressure and/or laser.

The system may include one or more robot arms, each of which may be mounted on any support structure such as a frame, table, chair, mobile cart, and so on. The fixture on which the user rests may be a therapy table, chair, frame, or any other similar fixture. Sensors conforming the sensor array may be mounted relative to the resting surface, mounted mobile relative to the resting surface, or any combination thereof. The sensor array may be replaced by a single sensor or a sensor module, and may use alternate sensing methods such as optical, lidar, magnetic, electro-magnetic, and so on.

The end-effector may be interchangeable with end-effectors of various designs. An end-effector may be designed to deliver one therapy modality or a combination of several modalities. A therapy modality may be used individually or combined with other therapy modalities at the same time (e.g., pressure and laser). The end-effector does not have to be used exclusively to deliver therapy. It may also be used to place and/or remove a number of pods that include appropriate apparatus to deliver the therapy. These pods can be wireless or wired. The pods may be temporarily attached to the user's skin via suction cups, weak adhesive, or any other method of temporary attachment.

The real-time biomechanical model may be of a similar kind as the user-scale model, which can be based on physics methods and geometric methods. 3D key-point localization may be done without previous occlusion removal, or by performing key-point detection on the 2D input images separately and then triangulating locations. The end effector may use acoustic waves as a therapy, such as in shockwave therapy (ESWT), or may use them in the form of ultrasound to assist in locating internal anatomy.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the subject disclosure.

What is claimed is:

1. A robotic system, comprising:
an end effector coupled to a distal end of a robotic arm, one or more cameras; and a processors configured to:
construct a three-dimensional (3D) model of a user based on received data from the one or more cameras;
identify automatically a target therapy point on the user based on the constructed 3D model; and
actuate the end effector to apply a therapy to the target therapy point.

2. The robotic system of claim 1 further comprises a table for the user to lay on during the therapy.

3. The robotic system of claim 1 further comprises a frame, wherein the robotic arm and the one or more cameras are mounted to the frame.

4. The robotic system of claim 1, wherein the one or more cameras includes color and/or depth cameras, and wherein the data received from the one or more cameras includes color images and/or a surface point cloud of the user.

5. The robotic system of claim 4, wherein identifying the target therapy point comprises:
generating a synthetic image of the user based on the color images received from the one or more cameras; and
identifying key-point locations on the synthetic image automatically using machine learning.

6. The robotic system of claim 5, wherein generating the synthetic image comprises removing occlusion from the color images received from the one or more cameras.

7. The robotic system of claim 4, wherein the 3D model constructed is a biomechanical model scaled to the specific size and morphology of the user, the biomechanical model comprising musculoskeletal geometry of the user.

8. The robotic system of claim 7, wherein the processor is further configured to track user motion in 3D space in real-time based on the surface point cloud and the 3D biomechanical model of the user.

9. The robotic system of claim 8, wherein identifying the target therapy point comprises mapping a key-point in an image to the 3D biomechanical model of the user.

10. The robotic system of claim 1, wherein the end effector is configured to deliver one or more of the therapy modalities, including pressure, vibration, heat, electricity, laser, acoustic waves, needling, moxibustion, and vacuum cupping.

11. A computer-implemented method, comprising:
receiving, by a processor, data about a user from one or more sensors;
constructing a three-dimensional (3D) model of the user based on the received data from the one or more sensors;
identifying, by the processor, a target point on the user based on the constructed 3D model; and
actuating an end effector to apply therapy to the target point.

12. The computer-implemented method of claim 11, wherein the data received from the one or more sensors includes color images and/or a surface point cloud of the user.

13. The computer-implemented method of claim 12, wherein identifying the target point comprises:
generating a synthetic image of the user based on the color images received from the one or more sensors; and
identifying key-point locations on the synthetic image automatically.

14. The computer-implemented method of claim 13, wherein generating the synthetic image comprises removing occlusion from the color images received from the one or more sensors.

15. The computer-implemented method of claim 12, wherein the 3D model constructed is a biomechanical model scaled to the specific size and morphology of the user, the biomechanical model comprising musculoskeletal geometry of the user.

16. The computer-implemented method of claim 15, further comprising tracking user motion in 3D space in real-time based on the surface point cloud and the 3D biomechanical model of the user.

17. The computer-implemented method of claim 11, wherein the end effector is configured to deliver one or more of the therapy modalities, including pressure, vibration, heat, electricity, laser, acoustic waves, needling, moxibustion, and vacuum cupping.

18. An apparatus, comprising:
one or more sensors; and
a control unit configured to:
construct a 3D model of a user based on received data from the one or more sensors;
identify a therapy point on the user based on the constructed 3D model; and
actuate an end effector to treat the target point.

19. The apparatus of claim 18, wherein the one or more sensors includes color and/or depth imaging sensors, and wherein the data received includes color images and/or a surface point cloud of the user.

20. The apparatus of claim 18, wherein the end effector is configured to deliver one or more of the therapy modalities, including pressure, vibration, heat, electricity, laser, acoustic waves, needling, moxibustion, and vacuum cupping.
